(19) United States
(12) Patent Application Publication
(10) Pub. No.: US 2005/0192478 A1
(43) Pub. Date: Sep. 1, 2005

(54) System and method for endoscopic optical constrast imaging using an endo-robot

(76) Inventors: James P. Williams, Princeton Junction, NJ (US); Klaus Abraham-Fuchs, Erlangen (DE)

Correspondence Address: Siemens Corporation;Intellectual Property Department, 170 Wood Avenue South, Iselin, NJ, 08830, US

(21) Appl. No.: 11/044,575

(22) Filed: Jan. 27, 2005

Related U.S. Application Data
(60) Provisional application No. 60/548,540, filed on Feb. 27, 2004.

(51) Int. Cl. A61B 1/06; A61M 25/00

(52) U.S. Cl. 600/160; 600/178; 600/476; 600/117

(57) ABSTRACT

A system and method for endoscopic optical imaging using an endo-robot is provided. The method for performing endoscopic optical imaging using a capsule endoscope comprises: navigating the capsule endoscope through a lumen of a patient that has been introduced with an optical contrast agent; illuminating a portion of the lumen that is not penetrable by an external light source with light emitted from the capsule endoscope to enhance an image intensity of the portion of the lumen; and powering the capsule endoscope with an externally applied magnetic field.

### CROSS-REFERENCE TO RELATED APPLICATION

This application claims the benefit of U.S. Provisional Application No. 60/548,540, filed Feb. 27, 2004, the disclosure of which is herein incorporated by reference.

### BACKGROUND OF THE INVENTION

1. Technical Field

The present invention relates to an endoscopic examination, and more particularly, to endoscopic examination using an endo-robot.

2. Discussion of the Related Art

An endoscopy is the examination and inspection of the interior of body organs, joints or cavities through an endoscope and has become a common practice in the medical diagnosis of many internal body diseases. The endoscope is a tubular device using fiber optics and a powerful lens system to provide lighting and visualization of the interior body organs, joints or cavities. During an endoscopy, the lens end or head of the endoscope is inserted into, for example, a gastrointestinal tract, and is moved along by an external pushing action. Because the movement of the endoscope is brought about by a pushing action, the impact of the lens end against a wall of the gastrointestinal tract can be discomforting to a patient. In addition, as the lens end enters a bend in the gastrointestinal tract, the wall can be damaged if too much force is applied. These hindrances typically limit the endoscopic examination to non-convoluted regions of the gastrointestinal tract.

In order to overcome the risks associated with the pushing action of a conventional endoscopy, a capsule endoscope has been developed. The capsule endoscope is typically introduced into a patient by swallowing the capsule endoscope to move the capsule endoscope from the esophagus through the stomach, the duodenum and subsequently the small intestine. During this time, the capsule endoscope captures images of the inside of the patient's body using, for example, a camera, an image pickup device and a light source sealed within the capsule endoscope. The capsule endoscope then wirelessly transmits the captured image signals to an analysis device such as a computer workstation outside the patient's body.

The capsule endoscope, however, does not have a self-advancing or position function. Thus, a medical practitioner who is analyzing data received from the capsule endoscope cannot control how and in which direction the capsule endoscope advances throughout the patient's body. In addition, an internal power supply powers the capsule endoscope, and supports the illumination, image acquisition and wireless transmission of data to an external receiver linked to the analysis device. Thus, because many images have to be acquired in order to cover the entire length of the gastrointestinal tract, a large amount of energy is consumed and in most cases drained before the entire tract can be examined.

In a Magnetic Resonance Imaging (MRI) or Computed Tomography (CT) scan, a contrast agent is often introduced into a patient. The contrast agent such as a dye is used to highlight specific areas of the patient so that organs, blood vessels, or tissues are more visible. In particular, by increasing the visibility of all surfaces of the organs or tissues being studied, contrast agents can help a medical practitioner determine the presence and extent of a disease or injury. Common contrast agents include compounds such as iodine, barium, barium sulfate or gastrografin. Contrast agents are also used in the practice of optical imaging as an optical contrast agent may illuminate when exposed to, for example, infrared (IR) light at certain wavelengths. However, when light is unable to penetrate to certain depths of the patient's body, the contrast agent is not illuminated and diseases or injuries that are in the un-illuminated region may not be detected and subsequently treated.

Accordingly, there is a need for an endoscopic examination technique that uses a capsule endoscope, which can be externally controlled and powered, and that can illuminate contrast agent treated regions of a patient's body that are not penetrated by light in a conventional manner.

### SUMMARY OF THE INVENTION

The present invention overcomes the foregoing and other problems encountered in the known teachings by providing a system and method for performing endoscopic optical imaging using an endo-robot.

In one exemplary embodiment of the present invention, a method for performing endoscopic optical imaging using a capsule endoscope is provided. The method comprising: navigating the capsule endoscope through a lumen of a patient that has been introduced with an optical contrast agent; illuminating a portion of the lumen that is not penetrable by an external light source with light emitted from the capsule endoscope to enhance an image intensity of the portion of the lumen; and powering the capsule endoscope with an externally applied magnetic field.

The method further comprises administering the optical contrast agent to the lumen of the patient. The optical contrast agent is administered by one of intravenous injection, oral administration, rectal administration, and inhalation. The optical contrast agent is one of indocyanine green, methelyne blue and flourescein. The lumen is one of a gastrointestinal tract, pancreas, bronchi, larynx, trachea, sinus, ear canal, blood vessel, urethra and bladder. The method further comprises inserting the capsule endoscope into the lumen of the patient. The capsule endoscope is inserted by one of oral insertion, rectal insertion, and through a sluice.

In another exemplary embodiment of the present invention, a method for treating a pathology using a capsule endoscope is provided. The method comprising: navigating the capsule endoscope through a lumen that has been introduced with a tumor-targeted contrast agent; identifying a tumor in the lumen made visible by the tumor-targeted contrast agent using a viewing device of the capsule endoscope; treating the tumor with phototherapy using an illumination device of the capsule endoscope; and powering the capsule endoscope with an externally applied magnetic field.

The tumor-targeted contrast agent is one of a metal nanopartical, quantum dot, and organic fluorescent dye. The tumor-targeted contrast agent is coupled to a monoclonal antibody to bind to a tumor. The tumor is one of a nodule, lesion, polyp, pre-cancerous growth, or cancerous growth. The phototherapy is one of ultraviolet light B (UVB) therapy, photochemotherapy, and photodynamictherapy.

In yet another exemplary embodiment of the present invention, a system for performing endoscopic optical imaging is provided. The system comprising: a capsule endoscope, comprising: a linear magnet for enabling the capsule endoscope to be moved inside a lumen of a patient; a camera for capturing images inside the lumen, an illuminator for illuminating the inside of the lumen; a controller for controlling the camera and the illuminator; a transceiver for performing one of transmitting the images captured by the camera and receiving commands from outside the patient; and a power supply for receiving an inductive charge from an externally applied magnetic field; and a magnetic control system, comprising: a first and second magnet tube for generating the magnetic field for controlling the movement of the capsule endoscope inside the lumen, a gradient amplifier for changing the direction of the magnetic field; a plurality of sensors for receiving location and orientation signals from the capsule endoscope; a location measuring device for processing the location and orientation signals from the capsule endoscope; and a control unit for controlling the movement of the capsule endoscope using the received location and orientation signals.

The linear magnet is a superconducting magnet. The illuminator is one of an infrared (IR) light emitting device, light emitting diode (LED), high-performance three-color LED, and micro-fluorescent lamp. The illuminator is capable of treating a tumor inside the lumen with phototherapy. The power supply is an accumulator.

The capsule endoscope further comprises: a drug delivery device for delivering drugs to a tumor inside the lumen; a biopsy gun for acquiring samples of a suspected pathological site inside the lumen; a surgical device for performing one of applying a mechanical force to a suspected pathological site inside the lumen to determine the elasticity of the suspected pathological site and for removing a tumor from the lumen; a sound generator for generating sound waves to be targeted at a suspected pathological site inside the lumen; and a measuring device for measuring one of temperature, electrical conductivity, pressure, and chemical levels inside the lumen.

The location measuring device is a transponder. The magnetic control system further comprises: a reception unit for receiving the captured images from the capsule endoscope and for transmitting the captured images to the control unit; and a bed for receiving the patient.

In another exemplary embodiment of the present invention, a method for performing an endoscopic examination using an endo-robot is provided. The method comprising: administering a contrast agent to a lumen of a patient; introducing the endo-robot into the lumen; navigating the endo-robot through the lumen; illuminating a portion of the lumen that is not penetrable by an external light source using the endo-robot; identifying a tumor in the portion of the lumen using the endo-robot; treating the tumor with phototherapy using the endo-robot; and powering the endo-robot with an externally applied magnetic field.

The foregoing features are of representative embodiments and are presented to assist in understanding the invention. It should be understood that they are not intended to be considered limitations on the invention as defined by the claims, or limitations on equivalents to the claims. Therefore, this summary of features should not be considered dispositive in determining equivalents. Additional features of the invention will become apparent in the following description, from the drawings and from the claims.

### BRIEF DESCRIPTION OF THE DRAWINGS

FIG. 1 is a block diagram of an endo-robot according to an exemplary embodiment of the present invention;

FIG. 2 is a block diagram of a system for performing endoscopic optical imaging using an endo-robot according to an exemplary embodiment of the present invention;

FIG. 3 is a flowchart illustrating a method for performing endoscopic optical imaging using an endo-robot according to an exemplary embodiment of the present invention; and

FIG. 4 is a flowchart illustrating a method for treating a pathology using an endo-robot according to an exemplary embodiment of the present invention.

### DETAILED DESCRIPTION OF EXEMPLARY EMBODIMENTS

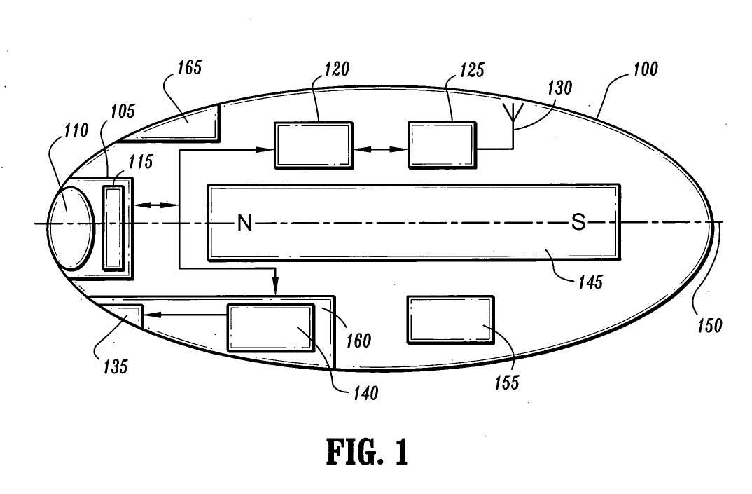

FIG. 1 is a block diagram of an endo-robot 100 according to an exemplary embodiment of the present invention. As shown in FIG. 1, the endo-robot 100 has an ellipsoidal housing in which a bar magnet 145 or a drivable approximately linear coil is co-linearly arranged about an axis 150. The endo-robot 100 includes a camera 105, for example, a video camera, having a lens 110 and an image sensor 115, for example, a charge coupled device (CCD) or complementary metal oxide semiconductor (CMOS) image sensor, for capturing images of the inside of a patient's body.

The endo-robot 100 also includes an illumination device 160, which includes an illuminator 135 and an illumination circuit 140. The illuminator 135 may be, for example, an infrared (IR) light emitting device, light emitting diode (LED), high-performance three-color LED or micro-fluorescent lamp for lighting an area surrounding the endo-robot 100 or for providing targeted phototherapy. Coupled to the camera 105 and the illumination device 160 is a control circuit 120 for controlling the operation of the endo-robot 100, and in particular, for controlling the rate of image acquisition of the camera 105 and the internal rotational movement or directional positioning of the lens 110 and illuminator 135.

As further shown in FIG. 1, the endo-robot 100 includes a transceiver 125, such as a radio frequency (RF) transceiver, and an antenna 130, both of which are coupled to the control circuit 120. The transceiver 125 and antenna 130 are used to transmit images acquired by the camera 105 to an external device for analysis. In addition, the transceiver 125 and antenna 130 can be used to receive commands from an external device to perform certain operations inside the patient's body. The endo-robot 100 further includes a power supply 155 such as an accumulator that can be inductively charged using an externally applied magnetic field. The endo-robot 100 could also include a small battery for use as a back-up power source.

The endo-robot 100 further includes a surgical/analysis portion 165 that could include a drug delivery mechanism for delivering drugs directly to a tumor or a biopsy gun for acquiring samples of a suspected pathological cite inside a lumen such as the gastrointestinal tract of a patient. In addition, the surgical/analysis portion 165 could include a tool arm or probe having, for example, knives, forceps, or snares, which could be used to apply a mechanical force to determine the elasticity of a suspected pathological site or a sound generator for generating sound waves to be targeted at the suspected pathological site. Further, the surgical/analysis portion 165 could include a measuring device having sensors for measuring temperature, electrical conductivity, pressure, and pH or other chemical levels inside the tract.

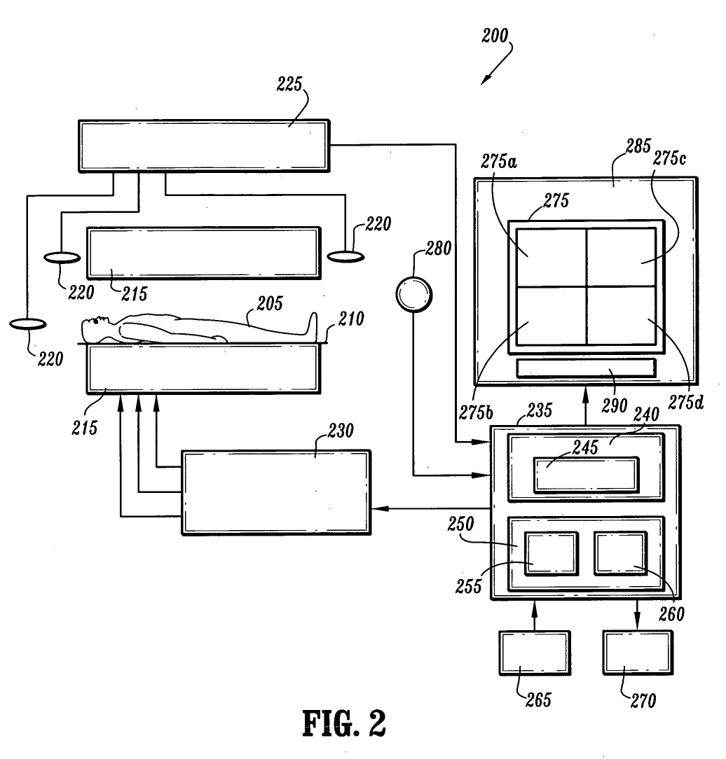

FIG. 2 is a block diagram of a system 200 for performing endoscopic optical imaging using an endo-robot according to an exemplary embodiment of the present invention. As shown in FIG. 2, the system 200 includes a pair of magnet tubes 215 and a bed 210 positioned on top of one of the tubes 215 on which a patient 205 may lie. The magnet tubes 215 have field coils for generating a stationary homogeneous magnetic field \vec{B}₀ as well as one gradient coil each coupled to an associated three-channel gradient amplifier 230. The three-channel gradient amplifier 230 is used to locally change the magnetic field in the ±x, ±y and ±z directions. The magnet tubes 215 and three-channel gradient amplifier 230 are used to control the endo-robot 100 as it travels through the gastrointestinal tract of the patient.

The system 200 also includes sensors 220 positioned around the patient 205 for picking up three-dimensional (3D) location and orientation signals transmitted from the endo-robot 100. The sensors 220, which may be, for example, antennas, transmit the location signals from the endo-robot 100 and forward them to a location-measuring device 225 such as a transponder. The location-measuring device 225 then forwards this data to a computer 235 for analysis. The system 200 further includes a reception unit 280, such as a transceiver, for receiving image data transmitted by the endo-robot 100 and for transmitting the image data to the computer 235.

As further shown in FIG. 2, the computer 235 includes a central processing unit (CPU) 240 and a memory 250, which are connected to an input 265 and output device 270. The CPU 240 includes a module 245 that includes one or more methods for performing endoscopic optical imaging using the endo-robot 100. The memory 250 includes a random access memory (RAM) 255 and a read only memory (ROM) 260. The memory 250 can also include a database, disk drive, tape drive, etc., or a combination thereof. The RAM 255 functions as a data memory that stores data used during execution of a program in the CPU 240 and is used as a work area. The ROM 260 functions as a program memory for storing a program executed in the CPU 240. The input 265 may be constituted by a keyboard, mouse, etc., and the output 270 may be constituted by a liquid crystal display (LCD), cathode ray tube (CRT) display, printer, etc.

The operation of the system 200 is controlled from an operator's console 285, which includes a controller 290, for example, a keyboard, and a display 275, for example, a CRT display. The operator's console 285 communicates with the computer 235, the pair of magnet tubes 215, the three-channel gradient amplifier 230 and the location-measuring device 225 to control the endo-robot 100. For example, the operator's console 285 can command the magnet tubes 215 to generate a static magnetic field for compensating the force of gravity on the endo-robot 100. This compensation of the force of gravity exerted on the endo-robot 100 makes it possible to move the endo-robot 100 in a free-floating manner in a lumen such as an intestine or a blood vessel. In particular, the magnetic field enables a linear force and a torque to be generated as long as the bar magnet 145 in the endo-robot 100 and the magnetic field are not co-linear. In addition to generating the torque, the steepness of the gradient of the magnetic field can also be used to define a translational force of the bar magnet 145.

As further shown in FIG. 2, when data is received from one of the sensors 220 or the reception unit 280 by the computer 235, the computer 235 may generate, for example, a three-sided view of the data by calculating virtual sectional views to be observed on the display 275. More specifically, the virtual sectional views may be calculated along sections parallel to the three orthogonal principal planes of the human body, for example, the sagittal, coronal, and transverse or axial planes, and can be displayed in three different control windows 275a-c of the display 275. In addition, an image sequence recorded by the endo-robot 100 can be played back in the form of a video in real-time in a fourth control window 275d of the display 275.

The operator's console 285 may further include any suitable image rendering system/tool/application that can process digital image data of an acquired image dataset (or portion thereof) to generate and display two-dimensional (2D) and/or 3D images on the display 275 using, for example, a 3D graphics card. More specifically, the image rendering system may be an application that provides 2D/3D rendering and visualization of image data, and which executes on a general purpose or specific computer workstation. The computer 235 may also include an image rendering system/tool/application for processing digital image data of an acquired image dataset to generate and display 2D and/or 3D images. In addition, it is to be understood that the computer 235 can be configured to operate and display information provided by the sensors 220 or the reception unit 280 absent the operator's console 285, using, for example, the input 265 and output 270 devices to execute certain tasks performed by the controller 290 and display 275.

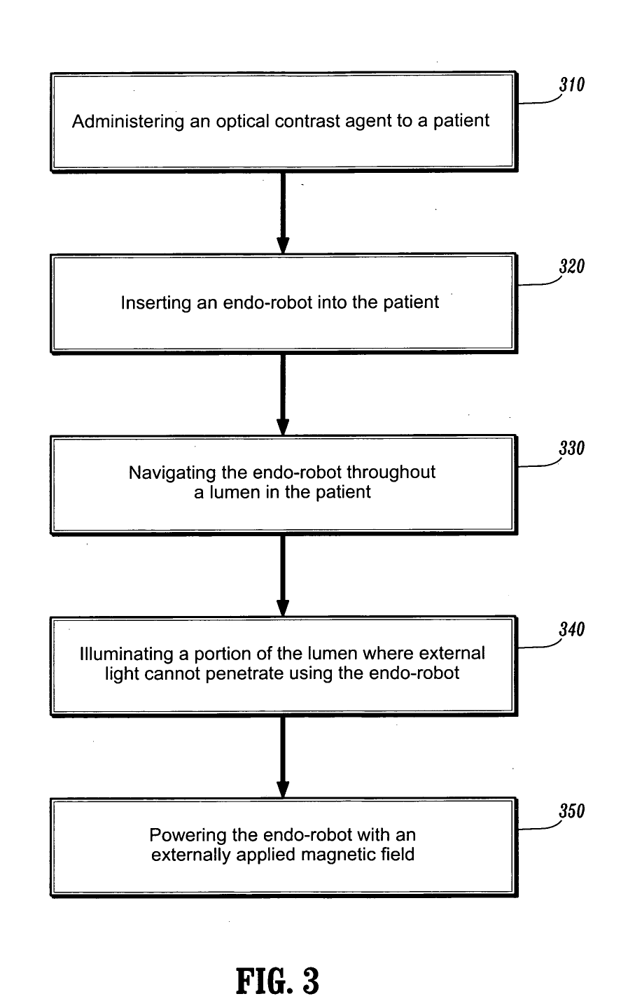

FIG. 3 is a flowchart illustrating a method for performing endoscopic optical imaging using an endo-robot according to an exemplary embodiment of the present invention. As shown in FIG. 3, a contrast agent is administered to a patient (310). In particular, an optical contrast agent such as indocyanine green, methelyne blue or fluorescein that glows when exposed to light at certain wavelengths is administered. It is to be understood, however, that any suitable optical contrast agent that glows when exposed to light may be administered in this step. The optical contrast agent may be administered in a number of ways such as through intravenous injection, oral or rectal administration, and inhalation.

After administering the optical contrast agent, the endo-robot 100 is inserted into the patient (320). The endo-robot 100 may be inserted simultaneously with the administration of the optical contrast agent, prior to or after the administration of the contrast agent depending on the technique used to administer the contrast agent. The endo-robot 100 is typically inserted into the patient by the patient swallowing the endo-robot 100 as it may come in a capsule form. It should also be understood that the endo-robot 100 could be inserted, for example, into the patient rectally or via a sluice.

Once the endo-robot 100 is in the patient, it can be navigated throughout the gastrointestinal tract of the patient (330). As discussed above with reference to FIGS. 1 and 2, the endo-robot 100 can be navigated by a user at the operator's console 285. For example, from the operator's console 285, the user can instruct the endo-robot 100 to transmit real-time images of the inside of the gastrointestinal tract of the patient. These images can be reproduced on the display 275 of the operator's console 285 thus enabling the user to analyze and inspect portions of the gastrointestinal tract as the endo-robot 100 passes through. By observing the inside of the gastrointestinal tract in real-time, the user can stop, rotate, reverse the course of, or aim the lens 105 or illuminator 135 of the endo-robot 100 to inspect regions of interest such as protrusions or polyp-like structures inside the tract that may indicate a pathology or injury in the tract.

As the endo-robot 100 reaches, for example, the small intestine, which has portions thereof that are not typically capable of being penetrated by an external light source, the endo-robot 100 is used to illuminate an area so that it can be observed (340). As a visible light source only penetrates up to 1-3 centimeters and a near-IR light source only penetrates up to 5-15 centimeters, an area that is not capable of being penetrated by an external light source may be an area that is more than or in some cases less than 15 centimeters from the surface of the patient.

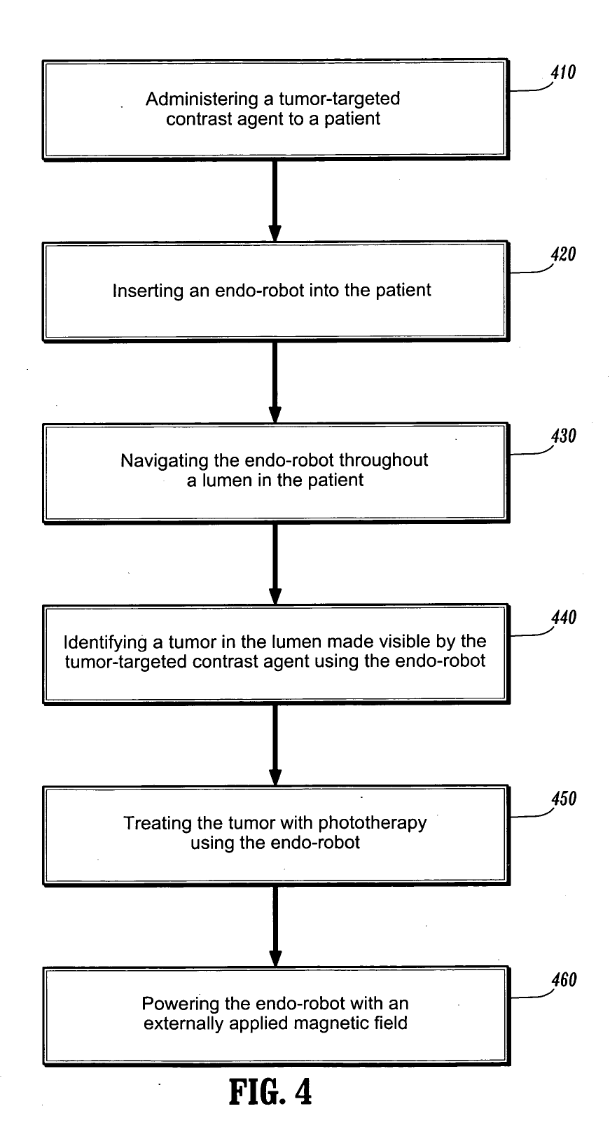

Upon reaching the small intestine, the endo-robot 100 can be instructed to, for example, increase the amount of light or modify the wavelength of the light emitted by its illuminator 135 to enhance the local image intensity of the optical contrast agent treated area being viewed. This allows the user to view a portion of the gastrointestinal tract that may have been previously un-observable. Further, this provides the user with a clearer image than may have been possible using a conventional illumination device. If, for example, a pathology is detected in this area, it can be observed and analyzed by the user in real-time and data that is associated with the pathology such as position, size and elasticity can be transmitted from the endo-robot 100 and stored in the memory 250 of the computer 235 for further analysis or use. In addition, if it can be determined that the pathology is, for example, a cancerous tumor, it can be treated with phototherapy in real-time or later by using the illumination device 160 to destroy the tumor. An example of the process of treating a tumor with phototherapy will be discussed with reference to FIG. 4.

In order to provide the endo-robot 100 with enough power to perform the above mentioned steps, the power supply 155 is charged using a magnetic field applied from the magnet tubes 215 (350). This is accomplished, for example, by commanding the magnet tubes 215 to power the power supply 155 through inductive charging upon receipt or determination of a low power indication from the endo-robot 100. It is to be understood, however, that the power supply 155 may be charged at any time the endo-robot 100 is inside the patient and before or after any of the steps previously discussed.

Although the process of treating a tumor with phototherapy to be discussed uses a tumor-targeted contrast agent and presumes that the location of the tumor is not known, it is to be understood that this process could be applied without a tumor-targeted contrast agent and that the location of the tumor is already known.

As shown in FIG. 4, a tumor-targeted contrast agent is administered to a patient (410). More specifically, any type of tumor-targeted contrast agent such as metal nanoparticals, quantum dots, and organic fluorescent dyes (e.g., fluorescein and indocyanine green) that can be used in conjunction with, for example, a monoclonal antibody, to bind to a tumor and that illuminates when exposed to light, is administered to the patient. The contrast agent may be administered to the patient in any of the manners as discussed above with reference to FIG. 3.

After administering the tumor-targeted contrast agent, the endo-robot 100 is inserted into the patient (420). Similar to insertion techniques discussed above with reference to FIG. 3, the endo-robot 100 may be inserted into the patient orally, rectally or via a sluice. Upon insertion, the endo-robot 100 is then navigated throughout a lumen of the patient (430) and using its camera 105 or illuminator 135 searches for a tumor that is made visible by the tumor-targeted contrast agent (440). Upon detection of a tumor in the lumen, the tumor is then treated with, for example, a phototherapy such as ultraviolet light B (UVB) therapy, photochemotherapy, and photodynamictherapy, using the endo-robot 100 (450). In particular, the illuminator 135 is aimed at the tumor and a wavelength of, for example IR light that is capable of destroying the tumor, is projected onto the tumor until it is destroyed.

In addition to treating the tumor with phototherapy in this step, the location of the tumor could be transmitted to a user at the operator's console 285 and this information could be used by a medical practitioner to perform a conventional ablation. Further, a surgical tool included in the endo-robot 100 could be extended therefrom to remove the tumor, or in the alternative, the tumor could be treated by injecting a drug carried by the endo-robot 100 into the tumor or sound waves that are capable of destroying the tumor could be emitted by the endo-robot 100. Also in this step, a drug that was previously injected into a patient and bound to a tumor could be bombarded with IR light from the endo-robot 100 to activate the drug and thus destroy the tumor.

As several of the procedures described above with reference to FIG. 4 require significant amounts of power, the power supply 155 of the endo-robot 100 must be externally charged using the magnet tubes 215 (460). This is accomplished using the techniques previously discussed with reference to FIG. 3.

Thus, in accordance with an exemplary embodiment of the present invention, an endo-robot is capable of performing an endoscopic examination that provides a medical practitioner with a complete viewing of a target area while reducing the risks and increasing the comfort of a conventional endoscopy. In addition, the present invention increases the duration of a conventional capsule endoscopy by providing the endo-robot with a power supply that is capable of being externally monitored and charged. Further, the present invention enables precise targeting and treatment of pathologies found during the examination by using, for example, phototherapy.

It is to be understood that because some of the constituent system components and method steps depicted in the accompanying figures may be implemented in software, the actual connections between the system components (or the process steps) may differ depending on the manner in which the present invention is programmed. Given the teachings of the present invention provided herein, one of ordinary skill in the art will be able to contemplate these and similar implementations or configurations of the present invention.

It is to be further understood that the present invention may be implemented in various forms of hardware, software, firmware, special purpose processors, or a combination thereof. In one embodiment, the present invention may be implemented in software as an application program tangibly embodied on a program storage device (e.g., magnetic floppy disk, RAM, CD ROM, DVD, ROM, and flash memory). The application program may be uploaded to, and executed by, a machine comprising any suitable architecture.

It should also be understood that the above description is only representative of illustrative embodiments. For the convenience of the reader, the above description has focused on a representative sample of possible embodiments, a sample that is illustrative of the principles of the invention. The description has not attempted to exhaustively enumerate all possible variations. That alternative embodiments may not have been presented for a specific portion of the invention, or that further undescribed alternatives may be available for a portion, is not to be considered a disclaimer of those alternate embodiments. Other applications and embodiments can be straightforwardly implemented without departing from the spirit and scope of the present invention.

It is therefore intended that the invention not be limited to the specifically described embodiments, because numerous permutations and combinations of the above and implementations involving non-inventive substitutions for the above can be created, but the invention is to be defined in accordance with the claims that follow. It can be appreciated that many of those undescribed embodiments are within the literal scope of the following claims, and that others are equivalent.

1. A method for performing endoscopic optical imaging using a capsule endoscope, comprising:
navigating the capsule endoscope through a lumen of a patient that has been introduced with an optical contrast agent;
illuminating a portion of the lumen that is not penetrable by an external light source with light emitted from the capsule endoscope to enhance an image intensity of the portion of the lumen; and
powering the capsule endoscope with an externally applied magnetic field.

2. The method of claim 1, further comprising:
administering the optical contrast agent to the lumen of the patient.

3. The method of claim 2, wherein the optical contrast agent is administered by one of intravenous injection, oral administration, rectal administration, and inhalation.

4. The method of claim 1, wherein the optical contrast agent is one of indocyanine green, methelyne blue and fluorescein.

5. The method of claim 1, wherein the lumen is one of a gastrointestinal tract, pancreas, bronchi, larynx, trachea, sinus, ear canal, blood vessel, urethra and bladder.

6. The method of claim 1, further comprising:
inserting the capsule endoscope into the lumen of the patient.

7. The method of claim 6, wherein the capsule endoscope is inserted by one of oral insertion, rectal insertion, and through a sluice.

8. A method for treating a pathology using a capsule endoscope, comprising:
navigating the capsule endoscope through a lumen that has been introduced with a tumor-targeted contrast agent;
identifying a tumor in the lumen made visible by the tumor-targeted contrast agent using a viewing device of the capsule endoscope;
treating the tumor with phototherapy using an illumination device of the capsule endoscope; and
powering the capsule endoscope with an externally applied magnetic field..

9. The method of claim 8, wherein the tumor-targeted contrast agent is one of a metal nanopartical, quantum dot, and organic fluorescent dye.

10. The method of claim 8, wherein the tumor-targeted contrast agent is coupled to a monoclonal antibody to bind to a tumor.

11. The method of claim 8, wherein the tumor is one of a nodule, lesion, polyp, pre-cancerous growth, or cancerous growth.

12. The method of claim 8, wherein the phototherapy is one of ultraviolet light B (UVB) therapy, photochemotherapy, and photodynamictherapy.

13. A system for performing endoscopic optical imaging, comprising:
a capsule endoscope, comprising: a linear magnet for enabling the capsule endoscope to be moved inside a lumen of a patient; a camera for capturing images inside the lumen, an illuminator for illuminating the inside of the lumen; a controller for controlling the camera and the illuminator; a transceiver for performing one of transmitting the images captured by the camera and receiving commands from outside the patient; and a power supply for receiving an inductive charge from an externally applied magnetic field; and
a magnetic control system, comprising: a first and second magnet tube for generating the magnetic field for controlling the movement of the capsule endoscope inside the lumen, a gradient amplifier for changing the direction of the magnetic field; a plurality of sensors for receiving location and orientation signals from the capsule endoscope; a location measuring device for processing the location and orientation signals from the capsule endoscope; and a control unit for controlling the movement of the capsule endoscope using the received location and orientation signals.

14. The system of claim 13, wherein the linear magnet is a superconducting magnet.

15. The system of claim 13, wherein the illuminator is one of an infrared (IR) light emitting device, light emitting diode (LED), high-performance three-color LED, and micro-fluorescent lamp.

16. The system of claim 13, wherein the illuminator is capable of treating a tumor in the lumen with phototherapy.

17. The system of claim 13, wherein the power supply is an accumulator.

18. The system of claim 13, wherein the capsule endoscope further comprises:
a drug delivery device for delivering drugs to a tumor inside the lumen.

19. The system of claim 13, wherein the capsule endoscope further comprises:
a biopsy gun for acquiring samples of a suspected pathological site inside the lumen.

20. The system of claim 13, wherein the capsule endoscope further comprises:
a surgical device for performing one of applying a mechanical force to a suspected pathological site inside the lumen to determine the elasticity of the suspected pathological site and for removing a tumor from the lumen.

21. The system of claim 13, wherein the capsule endoscope further comprises:
a sound generator for generating sound waves to be targeted at a suspected pathological site inside the lumen.

22. The system of claim 13, wherein the capsule endoscope further comprises:
a measuring device for measuring one of temperature, electrical conductivity, pressure, and chemical levels inside the lumen.

23. The system of claim 13, wherein the location measuring device is a transponder.

24. The system of claim 13, wherein the magnetic control system further comprises:
a reception unit for receiving the captured images from the capsule endoscope and for transmitting the captured images to the control unit.

25. The system of claim 13, wherein the magnetic control system further comprises:
a bed for receiving the patient.

26. A method for performing an endoscopic examination using an endo-robot, comprising:
administering a contrast agent to a lumen of a patient;
introducing the endo-robot into the lumen;
navigating the endo-robot through the lumen;
illuminating a portion of the lumen that is not penetrable by an external light source using the endo-robot;
identifying a tumor in the portion of the lumen using the endo-robot;
treating the tumor with phototherapy using the endo-robot; and
powering the endo-robot with an externally applied magnetic field.
